(19) United States
(12) Patent Application Publication
(10) Pub. No.: US 2026/0283947 A1
(43) Pub. Date: Sep. 24, 2026

(54) TOPICAL CREAM FOR TRANSDERMALLY APPLYING ESSENTIAL OILS FOR REDUCING PAIN, MUSCLE SPASMS, AND INFLAMMATION

(71) Applicant: Melanie Mitchell, Decatur, TX (US)

(72) Inventor: Melanie Mitchell, Decatur, TX (US)

(21) Appl. No.: 19/086,088

(22) Filed: Mar. 20, 2025

(51) Int. Cl. A61K 9/06; A61K 9/00; A61K 36/282; A61K 36/324; A61K 36/48; A61K 36/53; A61K 36/61; A61K 36/752; A61K 36/9066; A61K 47/12; A61K 47/44

(52) U.S. Cl. CPC A61K 9/06; A61K 9/0014; A61K 36/282; A61K 36/324; A61K 36/48; A61K 36/53; A61K 36/61; A61K 36/752; A61K 36/9066; A61K 47/12; A61K 47/44

(57) ABSTRACT

A cream for topical application that effectively delivers various active essential oils trans-dermally, and into the underlying tissues, including muscles, nerves, and blood vessels, while maintaining biological activity of the active essential oils to achieve reduction in perceived pain, reduced inflammation, and reduced muscle spasms. In certain aspects, this mixture includes waxes, oils, and butters that serve as lipophilic carriers of the active essential oils solubilized therein. Also disclosed is a method for making the cream for topical application.

### CROSS REFERENCE TO RELATED APPLICATIONS

This application claims priority from Provisional Patent Application 63/567,442, filed Mar. 20, 2024, the entire contents of which are hereby incorporated by reference.

### FIELD OF THE INVENTION

The present invention relates generally to topical creams, and particularly to topical creams for reducing pain, muscle spasms, and inflammation.

### BACKGROUND OF THE INVENTION

Users of topical creams for reducing pain, muscle spasms, and inflammation often encounter several issues. For example, many users report that topical creams, such as those containing NSAIDs (e.g., ibuprofen, diclofenac) or capsaicin, provide only partial or temporary relief. In fact, studies suggest that while NSAID creams can reduce pain in conditions like osteoarthritis or acute injuries, some users find no significant improvement.

For medicated creams (e.g., NSAID-based), users with certain conditions, such as ulcers, asthma, or kidney issues, or those on blood thinners, may experience risks similar to oral NSAIDs, such as gastrointestinal irritation or bleeding, though less common topically. Misuse or overapplication can increase these risks.

Other frequent complaints include skin-related side effects, such as redness, itching, burning, or rashes. Capsaicin-based creams, for example, can cause a burning sensation that some find intolerable, while products with menthol or salicylates may irritate sensitive skin or cause allergic reactions. Also. many topical creams, particularly those with menthol and/or camphor, have strong smells that some users find unpleasant or socially inconvenient.

### SUMMARY OF THE INVENTION

The topical cream of the invention for reducing pain, muscle spasms, and inflammation provides relief without many of the drawbacks of known topical creams available on the market. For example, the topical cream of the invention avoids the side effects of medicated creams (e.g., NSAID-based creams), such as gastrointestinal irritation or bleeding, as well as skin-related side effects, such as redness, itching, burning, irritation, allergic reactions, or rashes. Further, the topical cream of the invention has been formulated so as to emit scents that most users find pleasant for themselves and/or others.

One general aspect of the invention is a method for making a cream for topical application that effectively delivers various active essential oils trans-dermally, and into the underlying tissues. The method includes: mix wax/butter base ingredients to provide a wax and butter base, the wax and butter base ingredients including: emulsifying wax, steric acid, cocoa butter, shorea butter, and West African shea butter; melt the wax and butter base over a boiler on ‘low’ until the wax and butter base is fully melted; mix carrier oil base ingredients to provide a carrier oil base, the carrier oil base ingredients including: rosehip seed oil, argan oil, Douglas fir oil, and castor oil; mix the carrier oil base and the wax/butter base to provide a mixture of the carrier oil base and the wax/butter base that is substantially uniform; place the mixture of the carrier oil base and the wax/butter base in a freezer or refrigerator until the mixture cools to at least 40 degrees; mix active essential oils to provide an active essential oil mixture, the active essential oil ingredients including: blue tansy essential oil, blue yarrow essential oil, davana essential oil, copaiba balsam essential oil, curcuma zedoaria essential oil, clove essential oil, frankincense essential oil, rosemary essential oil, and bergamot citrus essential oil; add some of the active essential oils to the mixture of carrier oil base and wax/butter base after the mixture has cooled to at least 40 degrees; add some black castor oil; and stir the resulting mixture.

In some embodiments, the wax/butter base ingredients are mixed in a ratio substantially in accordance with: 20 grams of Emulsifying wax; 14 grams of Steric acid; 100 grams of Cocoa butter; 100 grams of Shorea butter; and 75 grams of West African Shea butter.

In some embodiments, the carrier oil base ingredients are mixed in a ratio substantially in accordance with: 50 grams of Rosehip seed oil; 50 grams of Argan oil; 50 grams of Douglas Fir oil; and 60 grams of Castor oil.

In some embodiments, the active essential oil mixture ingredients are mixed in a ratio substantially in accordance with: 2 oz Blue Tansy essential oil; 2 oz Blue Yarrow essential oil; 1 oz Davana essential oil; 2 oz Copaiba Balsam essential oil; 2 oz Curcuma Zedoaria essential oil; 2 oz Clove essential oil; 2 oz Frankincense essential oil; 2 oz Rosemary essential oil; and 2 oz Bergamot Citrus essential oil.

In some embodiments, after the step of: whisk the resulting mixture at low speed for two minutes while adding 2 tablespoons of arrow root powder, and before the step of add some Black Castor oil, further including the step of: whisk the resulting mixture at medium speed for a minute while adding at least one essential oil for additional scent.

In some embodiments, the at least one essential oil includes at least one of: lavender, eucalyptus, orange.

In some embodiments, after the steps of add some black castor oil and mix the resulting mixture, further comprising the steps of: use a spoon to fill ten 4 oz jars with the resulting mixture; and seal each jar until ready for use.

In some embodiments, after the step of: add some of the Active Essential Oils into the bowl with the mixture of carrier oil base and wax/butter base after it is cooled, and before the step of: add to the resulting mixture some black castor oil, further include the step of: whisk the resulting mixture at low speed while adding arrow root powder.

In some embodiments, after the step of: add some black castor oil; and before the step of: whisk the resulting mixture, further include the step of: add some drops of vitamin E.

In some embodiments, the step of stir the resulting mixture includes: whisk the resulting mixture at high speed for at least 4 minutes.

Another general aspect of the invention is a cream for topical application that effectively delivers various active essential oils trans-dermally, and into the underlying tissues. The cream is made by a method including: mix wax/butter base ingredients to provide a wax and butter base, the wax and butter base ingredients including: emulsifying wax, steric acid, cocoa butter, shorea butter, and West African shea butter; melt the wax and butter base over a boiler on ‘low’ until the wax and butter base is fully melted; mix carrier oil base ingredients to provide a carrier oil base, the carrier oil base ingredients including: rosehip seed oil, argan oil, Douglas fir oil, and castor oil; mix the carrier oil base and the wax/butter base to provide a mixture of the carrier oil base and the wax/butter base that is substantially uniform; place the mixture of the carrier oil base and the wax/butter base in a freezer or refrigerator until the mixture cools to at least 40 degrees; mix active essential oils to provide an active essential oil mixture, the active essential oil ingredients including: blue tansy essential oil, blue yarrow essential oil, davana essential oil, copaiba balsam essential oil, curcuma zedoaria essential oil, clove essential oil, frankincense essential oil, rosemary essential oil, and bergamot citrus essential oil; add some of the active essential oils to the mixture of carrier oil base and wax/butter base after the mixture has cooled to at least 40 degrees; add some black castor oil; and stir the resulting mixture.

In some embodiments, the wax/butter base ingredients are mixed in a ratio substantially in accordance with: 20 grams of Emulsifying wax; 14 grams of Steric acid; 100 grams of Cocoa butter; 100 grams of Shorea butter; and 75 grams of West African Shea butter.

In some embodiments, the carrier oil base ingredients are mixed in a ratio substantially in accordance with: 50 grams of Rosehip seed oil; 50 grams of Argan oil; 50 grams of Douglas Fir oil; and 60 grams of Castor oil.

In some embodiments, the active essential oil mixture ingredients are mixed in a ratio substantially in accordance with: 2 oz Blue Tansy essential oil; 2 oz Blue Yarrow essential oil; 1 oz Davana essential oil; 2 oz Copaiba Balsam essential oil; 2 oz Curcuma Zedoaria essential oil; 2 oz Clove essential oil; 2 oz Frankincense essential oil; 2 oz Rosemary essential oil; and 2 oz Bergamot Citrus essential oil.

In some embodiments, after the step of: whisk the resulting mixture at low speed for two minutes while adding 2 tablespoons of arrow root powder, and before the step of add some Black Castor oil, further comprising the step of: whisk the resulting mixture at medium speed for a minute while adding at least one essential oil for additional scent.

In some embodiments, the at least one essential oil includes at least one of: lavender, eucalyptus, orange.

In some embodiments, after the steps of add some black castor oil and mix the resulting mixture, further comprising the steps of: use a spoon to fill ten 4 oz jars with the resulting mixture; and seal each jar until ready for use.

In some embodiments, after the step of: add some of the Active Essential Oils into the bowl with the mixture of carrier oil base and wax/butter base after it is cooled, and before the step of: add to the resulting mixture some black castor oil, further comprising the steps of: whisk the resulting mixture at low speed while adding arrow root powder.

In some embodiments, after the step of: add some black castor oil; and before the step of: whisk the resulting mixture, further comprising the step of: add some drops of vitamin E.

In some embodiments, the step of stir the resulting mixture includes: whisk the resulting mixture at high speed for at least 4 minutes.

### BRIEF DESCRIPTION OF THE DRAWING

Many additional features and advantages will become apparent to those skilled in the art upon reading the following description, when considered in conjunction with the accompanying drawing, wherein:

FIG. 1 is a flow chart illustrating an embodiment of the method of the invention.

### DETAILED DESCRIPTION

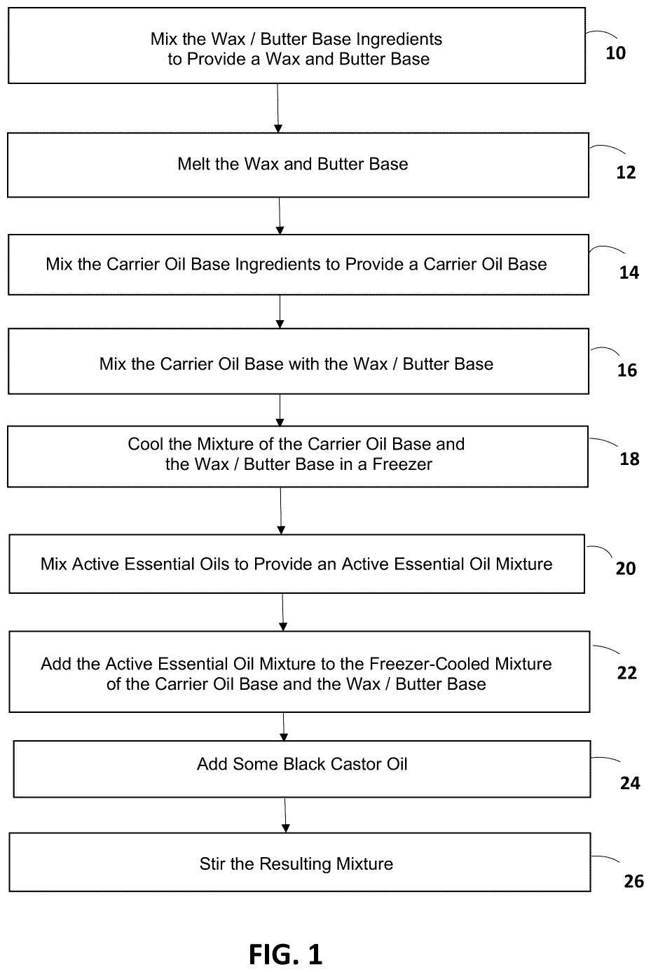

With reference to FIG. 1, to make the cream for topical application that effectively delivers various active essential oils trans-dermally, and into the underlying tissues:
First: Mix Wax/Butter Base 10:
Emulsifying wax 20 grams
Steric acid 14 grams
Cocoa butter 100 grams
Shorea butter 100 grams
West African Shea butter 75 grams

Note that the key to making this base Wax/Butter Base is the ratio of the various ingredients. Thus, the amounts can be scaled higher or lower, as long as the ratios are substantially maintained.

Melt the resulting mixture 12 over a double boiler on ‘low’ until fully melted. For melting delicate ingredients like wax and butter (which have relatively low melting points), “low” generally means a gentle heat to avoid scorching or overheating. On most standard stovetops, “low” corresponds to roughly 200° F. to 250° F. This range is sufficient to melt butter, which begins to soften around 90° F. (32° C.) and fully melts by 120° F., and most common waxes (like paraffin or beeswax), which melt between 120° F. to 160° F. (49° C. to 71° C.), depending on the type. Keeping the temperature in this lower range ensures a slow, even melt without breaking down the ingredients or causing separation. When using a double boiler, the water in the bottom pot is typically kept at a simmer on low heat, i.e., approximately 200° F. to 250° F. for the mixture above it.

Shorea butter is essentially Shorea robusta seed oil, which is an edible oil extracted from the seeds of the Shorea robusta tree. Shorea robusta is known as the Sal tree in India, where the tree is indigenous. The extracted crude Sal oil/fat is greenish-brown and has a distinctive scent. Due to the presence of more saturated fatty acids, it is solid at room temperature, and is therefore referred to as a fat or a butter.
Next: Mix Carrier Oil Base 14:
Rosehip seed oil 50 grams
Argan oil 50 grams
Douglas Fir oil 50 grams
Castor Oil 60 Grams

Note that the key to making this Carrier Oil Base is the ratio of the various ingredients. Thus, the amounts can be scaled higher or lower, as long as the ratios are substantially maintained.

Argan oil is made from the kernels of the argan tree (Argania spinosa L.).

Note that the steps of Mix Carrier Oil Base and Mix Wax/Butter Base can be performed in tandem, i.e., the order is not important.

Place the Carrier Oil Base and the Wax/Butter Base in a stainless steel mixing bowl (or glass bowl, for example), and stir 16 until substantially uniform.

Place the mixing bowl with the mixture of the Carrier Oil Base and the Wax/Butter Base in a freezer or refrigerator 18 for 20 minutes, or until the mixture cools to 40 degrees or lower.
Then: Mix Active Essential Oils 20: (for example, use a 24 oz container)
2 oz Blue Tansy essential oil
2 oz Blue Yarrow essential oil
1 oz Davana essential oil
2 oz Copaiba Balsam essential oil
2 oz Curcuma Zedoaria essential oil
2 oz Clove essential oil
2 oz Frankincense essential oil
2 oz Rosemary essential oil
2 oz Bergamot Citrus essential oil

Again, please note that the key to making this mixture of Active Essential Oils is the ratio of the various ingredients. Thus, the amounts can be scaled higher or lower, as long as the ratios are substantially maintained.

Also, the step of Mix Active Essential Oils can be performed while the mixture of the Carrier Oil Base and the Wax/Butter Base is cooling in the refrigerator or freezer.

Add two table spoons of the Active Essential Oils 22 into the bowl with the mixture of Carrier Oil Base and Wax/Butter Base after it is cooled.

Then, whisk the resulting mixture at low speed for two minutes while adding about 2 tablespoons of arrow root powder (to remove a greasy feeling when the final mixture is applied topically).

Optionally, whisk the resulting mixture at medium speed for a minute while adding about 3 oz of any desired essential oils for additional scent. Examples of such essential oils include: Lavender, Eucalyptus, Orange.

Next, add to the resulting mixture about ⅓ cup of Black Castor oil 24, stirring for about two minutes. Black Castor oil plays a supportive role by acting as a moisturizing carrier, aiding penetration, and enhancing the stability and duration of essential oil delivery into the skin and underlying tissues.

Then, add to the resulting mixture about 10 drops of Vitamin E, as a preservative.

Next, whisk the resulting mixture 26 at high speed for about 4 to 6 minutes, or until a desired texture is achieved.

Then, use a spoon to fill ten 4 oz jars (for example) with the resulting mixture, and seal each jar until ready for use.

Other modifications and implementations will occur to those skilled in the art without departing from the spirit and the scope of the invention as claimed. Accordingly, the above description is not intended to limit the invention, except as indicated in the following claims.

What is claimed is:

1. A method for making a cream for topical application that effectively delivers various active essential oils trans-dermally, and into the underlying tissues, the method comprising:
mix wax/butter base ingredients to provide a wax and butter base, the wax and butter base ingredients including: emulsifying wax, steric acid, cocoa butter, shorea butter, and West African shea butter;
melt the wax and butter base over a boiler on ‘low’ until the wax and butter base is fully melted;
mix carrier oil base ingredients to provide a carrier oil base, the carrier oil base ingredients including: rosehip seed oil, argan oil, Douglas fir oil, and castor oil;
mix the carrier oil base and the wax/butter base to provide a mixture of the carrier oil base and the wax/butter base that is substantially uniform;
place the mixture of the carrier oil base and the wax/butter base in a freezer or refrigerator until the mixture cools to at least 40 degrees;
mix active essential oils to provide an active essential oil mixture, the active essential oil ingredients including: blue tansy essential oil, blue yarrow essential oil, davana essential oil, copaiba balsam essential oil, curcuma zedoaria essential oil, clove essential oil, frankincense essential oil, rosemary essential oil, and bergamot citrus essential oil;
add some of the active essential oils to the mixture of carrier oil base and wax/butter base after the mixture has cooled to at least 40 degrees;
add some black castor oil; and
stir the resulting mixture.

2. The method of claim 1, wherein the wax/butter base ingredients are mixed in a ratio substantially in accordance with:
20 grams of Emulsifying wax;
14 grams of Steric acid;
100 grams of Cocoa butter;
100 grams of Shorea butter; and
75 grams of West African Shea butter.

3. The method of claim 1, wherein the carrier oil base ingredients are mixed in a ratio substantially in accordance with:
50 grams of Rosehip seed oil;
50 grams of Argan oil;
50 grams of Douglas Fir oil; and
60 grams of Castor oil.

4. The method of claim 1, wherein the active essential oil mixture ingredients are mixed in a ratio substantially in accordance with:
2 oz Blue Tansy essential oil;
2 oz Blue Yarrow essential oil;
1 oz Davana essential oil;
2 oz Copaiba Balsam essential oil;
2 oz Curcuma Zedoaria essential oil;
2 oz Clove essential oil;
2 oz Frankincense essential oil;
2 oz Rosemary essential oil; and
2 oz Bergamot Citrus essential oil.

5. The method of claim 1, after the step of:
whisk the resulting mixture at low speed for two minutes while adding 2 tablespoons of arrow root powder, and
before the step of add some Black Castor oil, further comprising the step of:
whisk the resulting mixture at medium speed for a minute while adding at least one essential oil for additional scent.

6. The method of claim 5, wherein the at least one essential oil includes at least one of: lavender, eucalyptus, orange.

7. The method of claim 1, after the steps of add some black castor oil and mix the resulting mixture, further comprising the steps of:
use a spoon to fill ten 4 oz jars with the resulting mixture; and
seal each jar until ready for use.

8. The method of claim 1, after the step of:
add some of the Active Essential Oils into the bowl with the mixture of carrier oil base and wax/butter base after it is cooled, and
before the step of: add to the resulting mixture some black castor oil, further comprising the step of:
whisk the resulting mixture at low speed while adding arrow root powder.

9. The method of claim 1, after the step of: add some black castor oil; and before the step of: whisk the resulting mixture, further comprising the step of:
add some drops of vitamin E.

10. The method of claim 1, wherein the step of stir the resulting mixture includes:
whisk the resulting mixture at high speed for at least 4 minutes.

11. A cream for topical application that effectively delivers various active essential oils trans-dermally, and into the underlying tissues, the cream made by a method comprising:
mix wax/butter base ingredients to provide a wax and butter base, the wax and butter base ingredients including: emulsifying wax, steric acid, cocoa butter, shorea butter, and West African shea butter;
melt the wax and butter base over a boiler on ‘low’ until the wax and butter base is fully melted;
mix carrier oil base ingredients to provide a carrier oil base, the carrier oil base ingredients including: rosehip seed oil, argan oil, Douglas fir oil, and castor oil;
mix the carrier oil base and the wax/butter base to provide a mixture of the carrier oil base and the wax/butter base that is substantially uniform;
place the mixture of the carrier oil base and the wax/butter base in a refrigerator or a freezer until the mixture cools to at least 40 degrees;
mix active essential oils to provide an active essential oil mixture, the active essential oil ingredients including: blue tansy essential oil, blue yarrow essential oil, davana essential oil, copaiba balsam essential oil, curcuma zedoaria essential oil, clove essential oil, frankincense essential oil, rosemary essential oil, and bergamot citrus essential oil;
add some of the active essential oils to the mixture of carrier oil base and wax / butter base after the mixture has cooled to at least 40 degrees;
add some black castor oil; and
stir the resulting mixture.

12. The cream for topical application of claim 11, wherein the wax/butter base ingredients are mixed in a ratio substantially in accordance with:
20 grams of Emulsifying wax;
14 grams of Steric acid;
100 grams of Cocoa butter;
100 grams of Shorea butter; and
75 grams of West African Shea butter.

13. The cream for topical application of claim 11, wherein the carrier oil base ingredients are mixed in a ratio substantially in accordance with:
50 grams of Rosehip seed oil;
50 grams of Argan oil;
50 grams of Douglas Fir oil; and
60 grams of Castor oil.

14. The cream for topical application of claim 11, wherein the active essential oil mixture ingredients are mixed in a ratio substantially in accordance with:
2 oz Blue Tansy essential oil;
2 oz Blue Yarrow essential oil;
1 oz Davana essential oil;
2 oz Copaiba Balsam essential oil;
2 oz Curcuma Zedoaria essential oil;
2 oz Clove essential oil;
2 oz Frankincense essential oil;
2 oz Rosemary essential oil; and
2 oz Bergamot Citrus essential oil.

15. The cream for topical application of claim 11, after the step of:
whisk the resulting mixture at low speed for two minutes while adding 2 tablespoons of arrow root powder, and
before the step of add some Black Castor oil, further comprising the step of:
whisk the resulting mixture at medium speed for a minute while adding at least one essential oil for additional scent.

16. The cream for topical application of claim 15, wherein the at least one essential oil includes at least one of: lavender, eucalyptus, orange.

17. The cream for topical application of claim 11, after the steps of add some black castor oil and mix the resulting mixture, further comprising the steps of:
use a spoon to fill ten 4 oz jars with the resulting mixture; and
seal each jar until ready for use.

18. The cream for topical application of claim 11, after the step of:
add some of the Active Essential Oils into the bowl with the mixture of carrier oil base and wax/butter base after it is cooled, and
before the step of: add to the resulting mixture some black castor oil, further comprising the step of:
whisk the resulting mixture at low speed while adding arrow root powder.

19. The cream for topical application of claim 11, after the step of: add some black castor oil; and before the step of: whisk the resulting mixture, further comprising the step of:
add some drops of vitamin E.

20. The cream for topical application of claim 11, wherein the step of stir the resulting mixture includes:
whisk the resulting mixture at high speed for at least 4 minutes.
